(19) United States
(12) Patent Application Publication
(10) Pub. No.: US 2022/0316008 A1
(43) Pub. Date: Oct. 6, 2022

(54) COMPOSITIONS COMPRISING RARE GENETIC SEQUENCE VARIANTS ASSOCIATED WITH PULMONARY FUNCTION AND METHODS OF USE THEREOF FOR DIAGNOSIS AND TREATMENT OF ASTHMA IN AFRICAN AMERICAN PATIENTS

(71) Applicant: THE CHILDREN'S HOSPITAL OF PHILADELPHIA, Philadelphia, PA (US)

(72) Inventors: Hakon Hakonarson, Malvern, PA (US); Patrick Sleiman, Philadelphia, PA (US)

(73) Assignee: THE CHILDREN'S HOSPITAL OF PHILADELPHIA, Philadelphia, PA (US)

(21) Appl. No.: 17/641,502

(22) PCT Filed: Sep. 8, 2020
(86) PCT No.: PCT/US2020/049773

§ 371 Date: Mar. 9, 2022

Related U.S. Application Data
(60) Provisional application No. 62/897,607, filed on Sep. 9, 2019.

(51) Int. Cl. C12Q 1/6883; A61P 11/06; A61K 31/5585; A61K 31/135

(52) U.S. Cl. CPC C12Q 1/6883; A61P 11/06; A61K 31/5585; A61K 31/135; C12Q 2600/156

(57) ABSTRACT

Compositions for the diagnosis and treatment of asthma are disclosed. In accordance with the present invention, a method for detecting, diagnosing and/or treating asthma in a human subject of African descent is provided. An exemplary method comprises detecting at least one single nucleotide polymorphism (SNP) listed in Table I or a SNP in linkage disequilibrium with one or more of the SNPs selected from rs2529168, rs2529136, rs2429063, rs2529155, 7:21303293, rs2700292, rs2700296, 7:21328865, rs10267234, and rs150512506 or a SNP in LD with any of said SNPs, in a nucleic acid sample from the subject, wherein detection is correlated with an increased risk, susceptibility, or predisposition to asthma.

This application claims priority to U.S. Provisional Application No. 62/897,607 filed Sep. 9, 2019, the entire contents being incorporated herein by reference as though set forth in full.

### FIELD OF THE INVENTION

The present invention relates to the fields of airway disease and genetic testing. More specifically, the invention provides compositions and methods for the diagnosis and treatment of asthma and other allergic conditions.

### BACKGROUND OF THE INVENTION

Several publications and patent documents are cited throughout the specification in order to describe the state of the art to which this invention pertains. Each of these citations is incorporated by reference herein as though set forth in full.

Asthma is a chronic inflammatory condition of the lungs characterized by excessive responsiveness of the lungs to stimuli in the forms of infections, allergens, and environmental irritants. Due to the variability of the disease and lack of generally agreed-on standards for diagnosis, it can be difficult to estimate the prevalence of asthma. Further, variations in practice from country to country complicate worldwide estimates. In the USA, it is estimated that at least 22.9 million Americans suffer from the condition.

Asthma is the leading chronic illness in US children. It is estimated that 300 million individuals suffer from asthma worldwide, with increased prevalence in both adults and children in recent decades. Prevalence is rising in locations where rates were previously low and variation in rates from country to country appears to be diminishing. Twin studies have shown that there is a genetic element to asthma susceptibility, with heritability of the condition estimated at between 36% and 77%. Since the publication of the first study linking a genetic locus to asthma in 1989, more than 100 candidate genes have been reported in connection to asthma or asthma-related phenotypes such as bronchial hyperresponsiveness and elevated levels of serum immunoglobulin (Ig) E. Initial studies were usually candidate-gene analyses, examining the role of specific loci in asthma in a hypothesis-based manner. A few loci were identified in a hypothesis-independent manner through traditional linkage analysis. These studies have elucidated several themes in the biology and pathogenesis of these diseases. The majority of these studies have been carried out in patients of European descent. Thus, there is still great uncertainty as to those genetic factors which contribute to early onset asthma in African Americans, where the disorder is quite prevalent.

### SUMMARY OF THE INVENTION

In accordance with the present invention, a method for detecting, diagnosing and/or treating asthma in a human subject of African descent is provided. An exemplary method comprises detecting at least one single nucleotide polymorphism (SNP) listed in Table 1 or a SNP in linkage disequilibrium with one or more of the SNPs selected from rs2529168, rs2529136, rs2429063, rs2529155, 7:21303293, rs2700292, rs2700296, 7:21328865, rs10267234, and rs150512506 or a SNP in LD with any of said SNPs, in a nucleic acid sample from the subject, wherein detection is correlated with an increased risk, susceptibility, or predisposition to asthma. The SNPs of Table 1 may be referred to herein as “asthma-associated single nucleotide polymorphisms (SNPs)”. The method can also entail diagnosing a subject with asthma if at least one asthma-associated SNP, or a SNP in linkage disequilibrium with one or more of the asthma-associated SNPs is detected, and optionally, administering an effective amount of one or more agents useful for the treatment of asthma. In certain embodiments, 1, 2, 3, 4, 5, 6, 7, or all of the SNPs in Table 1 are detected.

An exemplary method comprises detecting at least one single nucleotide polymorphism (SNP) listed in Table 2 or a SNP in linkage disequilibrium with one or more of the SNPs selected from rs12299028, rs192852410, rs145064303, rs189759151, rs181086557, rs142816400, rs144961519, rs78046756, rs116513973, rs115656979, rs147019971, rs74102922, rs74102924, rs74102926, rs74102933, rs74585484 or a SNP in LD with any of said SNPs, in a nucleic acid sample from the subject, wherein detection is correlated with an increased risk, susceptibility, or predisposition to asthma. The SNPs of Table 2 may be referred to herein as “asthma-associated single nucleotide polymorphisms (SNPs)”. The method can also entail diagnosing a subject with asthma if at least one asthma-associated SNP, or a SNP in linkage disequilibrium with one or more of the asthma-associated SNPs is detected, and optionally, administering an effective amount of one or more agents useful for the treatment of asthma. In certain embodiments, 1, 2, 3, 4, 5, 6, 7, or all of the SNPs in Table 2 are detected. Exemplary agents useful in the treatment of asthma are listed in Table 3.

The invention also provides cell lines comprising cilia obtained from patients which are homozygous (no risk alleles), heterozygous (one risk allele, one normal allele) and homozygous (two risk alleles). These cell lines can be used to advantage in screening assays to identify agents which modulate cilia function and activity.

In another embodiment, a method for diagnosing asthma in a human subject of African American ancestry is disclosed. An exemplary method comprises obtaining a nucleic acid sample from said subject; detecting whether the sample contains at least one asthma-associated single nucleotide polymorphism (SNP) such as any one or more of those listed in Tables 1 and 2, or a SNP in linkage disequilibrium with one or more of the asthma-associated SNPs, by contacting the nucleic acid sample with a probe or primer of sufficient length and composition to detect said SNP and diagnosing the subject as having asthma when the presence of at least one asthma-associated SNP, or a SNP in linkage disequilibrium with one or more of the asthma-associated SNP, in the nucleic acid sample is detected.

Kits for practicing the methods described above are also provided.

### BRIEF DESCRIPTION OF THE DRAWINGS

FIG. 1: A regional association plot showing novel locus at chromosome 7p15.3, identified in a large scale asthma meta-analysis of pediatric patients of African descent.

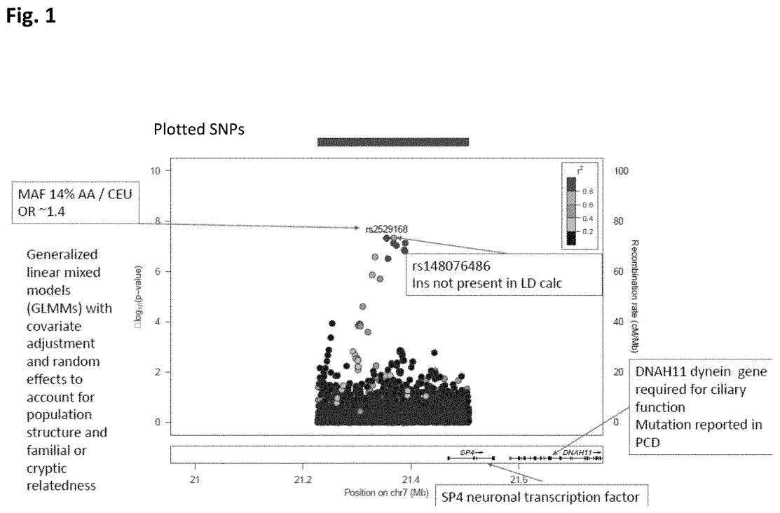

FIG. 2: A regional association plot for the IL22/IL26/Interferon-gamma asthma locus on chromosome 12, showing association with the sentinel SNPs and location in relation to IL22 and IL26. A table listing MAFs is provided in Example II. The top SNP maps to interferon gamma antisense ncRNA which could be used to advantage for diagnostic purposes.

### DETAILED DESCRIPTION OF THE INVENTION

As complex common diseases, asthma and allergic diseases are caused by the interaction of multiple genetic variants with a variety of environmental factors. Candidate-gene studies have examined the involvement of a very large list of genes in asthma and allergy, demonstrating a role for more than 100 loci. These studies have elucidated several themes in the biology and pathogenesis of these diseases. A small number of genes have been associated with asthma or allergy through traditional linkage analyses. The publication of the first asthma-focused genome-wide association (GWA) study in 2007 has been followed by nearly 30 reports of GWA studies targeting asthma, allergy, or associated phenotypes and quantitative traits. GWA studies have confirmed several candidate genes and have identified new, unsuspected, and occasionally uncharacterized genes as asthma susceptibility loci.

Dyneins are microtubule-associated motor protein complexes composed of several heavy, light, and intermediate chains. The axonemal dyneins, found in cilia and flagella, are components of the outer and inner dynein arms attached to the peripheral microtubule doublets. DNAH11 is a putative axonemal outer dynein arm heavy chain. Full-length DHAH11 contains 4,523 amino acids. DHAH11 has an N-terminal domain, followed by 4 AAA domains, a helix-1-MTB-helix-2 domain, 2 additional AAA domains, and a C-terminal domain containing a conserved GVALL motif. Each of the first 4 AAA domains contains a P-loop motif predicted to mediate ATP hydrolysis. The helix-1-MTB-helix-2 domain is predicted to interact with a microtubule. The present inventors have identified rare variants in DNAH11 which are associated with risk and/or development of asthma in African Americans.

In additional studies, the sentinel SNPs and their chromosomal location on chromosome 12 in relation to IL22, IL26 and interferon gamma loci have been identified which are predictive of risk of and predisposition to asthma in African Americans.

### Definitions

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu.

As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

“Asthma-associated SNP or specific marker” is a SNP or marker which is associated with an increased or decreased risk of developing asthma and found in lesser frequency in normal subjects who do not have this disease. Such markers may include but are not limited to nucleic acids, proteins encoded thereby, or other small molecules.

A “single nucleotide polymorphism (SNP)” refers to a change in which a single base in the DNA differs from the usual base at that position. These single base changes are called SNPs or “snips.” Millions of SNP's have been cataloged in the human genome. Some SNPs such as that which causes sickle cell are responsible for disease. Other SNPs are normal variations in the genome.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

“Linkage” describes the tendency of genes, alleles, loci or genetic markers to be inherited together as a result of their location on the same chromosome, and is measured by percent recombination (also called recombination fraction, or .theta.) between the two genes, alleles, loci or genetic markers. The closer two loci physically are on the chromosome, the lower the recombination fraction will be. Normally, when a polymorphic site from within a disease-causing gene is tested for linkage with the disease, the recombination fraction will be zero, indicating that the disease and the disease-causing gene are always co-inherited. In rare cases, when a gene spans a very large segment of the genome, it may be possible to observe recombination between polymorphic sites on one end of the gene and causative mutations on the other. However, if the causative mutation is the polymorphism being tested for linkage with the disease, no recombination will be observed.

“Centimorgan” is a unit of genetic distance signifying linkage between two genetic markers, alleles, genes or loci, corresponding to a probability of recombination between the two markers or loci of 1% for any meiotic event.

“Linkage disequilibrium” or “allelic association” means the preferential association of a particular allele, locus, gene or genetic marker with a specific allele, locus, gene or genetic marker at a nearby chromosomal location more frequently than expected by chance for any particular allele frequency in the population. Once a known SNP is identified, SNPs in linkage disequilibrium (also termed LD) may be identified via commercially available programs. For example, on the world wide web at analysistools.nci.nih.gov/LDlink/?tab=ldproxy. First, the LDproxy tab is selected. The reference rs number is entered, the r2 tab and the population of interest are selected and the SNPs in LD identified upon clicking on the “calculate” tab. A plot of surrounding area is revealed and a table with the SNPs in LD (with r2 values) is shown.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

“Target nucleic acid” as used herein refers to a previously defined region of a nucleic acid present in a complex nucleic acid mixture wherein the defined wild-type region contains at least one known nucleotide variation which may or may not be associated with asthma. The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated. Thus the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any asthma specific marker nucleic acid, but does not hybridize to other nucleotides. Also polynucleotide which “specifically hybridizes” may hybridize only to an airway specific marker, such as an asthma-specific marker shown in the Tables contained herein. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tm=81.5° C+16.6Log[Na+]+0.41(% G+C)−0.63(% formamide)−600/#bp in duplex.

As an illustration of the above formula, using [Na+]=[0.368] and 50% formamide, with GC content of 42% and an average probe size of 200 bases, the Tm is 57° C. The Tm of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tm of the hybrid. Wash conditions should be as stringent as possible for the degree of identity of the probe for the target. In general, wash conditions are selected to be approximately 12-20° C. below the Tm of the hybrid. In regards to the nucleic acids of the current invention, a moderate stringency hybridization is defined as hybridization in 6× SSC, 5× Denhardt's solution, 0.5% SDS and 100 μg/ml denatured salmon sperm DNA at 42° C., and washed in 2× SSC and 0.5% SDS at 55° C. for 15 minutes. A high stringency hybridization is defined as hybridization in 6× SSC, 5× Denhardt's solution, 0.5% SDS and 100 pg/ml denatured salmon sperm DNA at 42° C., and washed in 1× SSC and 0.5% SDS at 65° C. for 15 minutes. A very high stringency hybridization is defined as hybridization in 6× SSC, 5× Denhardt's solution, 0.5% SDS and 100 pg/ml denatured salmon sperm DNA at 42° C., and washed in 0.1× SSC and 0.5% SDS at 65° C. for 15 minutes.

The term “oligonucleotide,” as used herein is defined as a nucleic acid molecule comprised of two or more ribo or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20, at least about 30, at least about 40 or about 50 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single stranded or double stranded. The exact length of the probe will depend upon many factors, including temperature, source of probe and use of the method. For example, for diagnostic applications, depending on the complexity of the target sequence, the oligonucleotide probe typically contains 10, 15-25, 30, 50 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single stranded or double stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 10, 15-25, 30, 50 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template primer complex for the synthesis of the extension product. Polymerase chain reaction (PCR) has been described in U.S. Pat. Nos. 4,683,195, 4,800,195, and 4,965,188, the entire disclosures of which are incorporated by reference herein.

An “siRNA” refers to a molecule involved in the RNA interference process for a sequence-specific post-transcriptional gene silencing or gene knockdown by providing small interfering RNAs (siRNAs) that has homology with the sequence of the targeted gene. Small interfering RNAs (siRNAs) can be synthesized in vitro or generated by ribonuclease III cleavage from longer dsRNA and are the mediators of sequence-specific mRNA degradation. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif., USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting DENN/D1B mRNA, for example, may be between 15-35 nucleotides in length, and more typically about 21 nucleotides in length. Exemplary siRNA sequences effective for down-modulating expression of the asthma associated genes can be readily obtained from the above identified commercial sources.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the asthma specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the asthma specific marker encoding nucleic acid. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15, greater than 20 nucleotides long or greater than 30 nucleotides long.

“Sample” or “patient sample” or “biological sample” generally refers to a sample which may be tested for a particular molecule, preferably an asthma specific marker molecule, such as a marker shown in the tables provided below. Samples may include but are not limited to cells, body fluids, including blood, serum, plasma, urine, saliva, tears, pleural fluid and the like.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the SNP containing nucleic acids described herein or their encoded proteins. Agents are evaluated for potential biological activity by inclusion in screening assays described hereinbelow.

Methods of Using Asthma-Associated SNPS in DNAH11 for Diagnosing a Propensity for the Development of Asthma in Subjects of African American Ancestry

Nucleotides comprising asthma-associated single nucleotide polymorphisms (SNPs) as described herein in the Tables, for example at Table 1, may be used for a variety of purposes in accordance with the present invention. For example, asthma-associated SNP-containing DNA, RNA, or fragments thereof may be used as probes or primers to detect the presence of and/or expression of asthma-associated SNPs, or SNPs in linkage disequilibrium with one or more of the asthma-associated SNPs. Methods in which SNP-containing nucleic acids may be utilized as probes or primers include, but are not limited to: (1) in situ hybridization; (2) Southern hybridization (3) northern hybridization; and (4) assorted amplification reactions such as polymerase chain reactions (PCR) or quantitative PCR (qPCR).

Further, assays for detecting asthma-associated SNPs or the proteins encoded thereby may be conducted on any type of biological sample, including but not limited to body fluids (including blood, bronchial lavage, sputum, serum, gastric lavage, urine), any type of cell (such as brain cells, white blood cells, lung cells, fibroblast cells, mononuclear cells) or body tissue.

From the foregoing discussion, it can be seen that asthma-associated SNP containing nucleic acids, vectors expressing the same, asthma-associated SNP containing marker proteins and anti-asthma specific marker antibodies may be used to detect asthma associated SNPs in body tissue, cells, or fluid, and to diagnose, detect, or identify a human subject as having a predisposition for, or having, asthma.

In some embodiments for screening for asthma-associated SNPs, the asthma-associated SNP containing nucleic acid in the sample will initially be amplified, e.g. using PCR, to increase the amount of the templates as compared to other sequences present in the sample. This allows the target sequences to be detected with a high degree of sensitivity if they are present in the sample. This initial step may be avoided by using highly sensitive array techniques that are becoming increasingly important in the art.

Alternatively, new detection technologies can overcome this limitation and enable analysis of small samples containing as little as 1 μ66g of total RNA. Using Resonance Light Scattering (RLS) technology, as opposed to traditional fluorescence techniques, multiple reads can detect low quantities of mRNAs using biotin labeled hybridized targets and anti-biotin antibodies. Another alternative to PCR amplification involves planar wave guide technology (PWG) to increase signal-to-noise ratios and reduce background interference. Both techniques are commercially available from Qiagen Inc. (USA).

Thus any of the aforementioned techniques may be used to detect or quantify asthma-associated SNP marker expression and accordingly, diagnose asthma. Kits and Articles of Manufacture

Any of the aforementioned SNP-containing nucleic acids can be incorporated into a kit. In some embodiments, the kit comprises one or more nucleic acid molecules comprising an asthma-associated SNP. In some embodiments, the nucleic acid molecule is immobilized on a solid support, such as on a Gene Chip. In some embodiments, the solid support is affixed to the support so that it does not diffuse from the support when placed in solution. In some embodiments, the kit further comprises an oligonucleotide, a polypeptide, a peptide, an antibody, a label, marker, or reporter, a pharmaceutically acceptable carrier, a physiologically acceptable carrier, instructions for use, a container, a vessel for administration, an assay substrate, or any combination thereof.

Methods of Using Asthma-Associated SNPS for Development of Therapeutic Agents

Since the SNPs identified herein have been associated with the etiology of asthma, methods for identifying agents that modulate the activity of the genes and their encoded products containing such SNPs should result in the generation of efficacious therapeutic agents for the treatment of this condition.

DNAH11 protein coding regions along with the IL22, IL 26 and IFNgamma protein coding locus provide suitable targets for the rational design of therapeutic agents which modulate the activity of these proteins. Small peptide molecules corresponding to these regions may be used to advantage in the design of therapeutic agents which effectively modulate the activity of the encoded proteins.

Molecular modeling should facilitate the identification of specific organic molecules with capacity to bind to the active site of the proteins encoded by the SNP containing nucleic acids based on conformation or key amino acid residues required for function. A combinatorial chemistry approach will be used to identify molecules with greatest activity and then iterations of these molecules will be developed for further cycles of screening. In certain embodiments, candidate drugs can be screened from large libraries of synthetic or natural compounds. One example is an FDA approved library of compounds that can be used by humans. In addition, compound libraries are commercially available from a number of companies including but not limited to Maybridge Chemical Co. (Trevillet, Cornwall, UK), Comgenex (Princeton, N.J.), Microsource (New Milford, Conn.), Aldrich (Milwaukee, Wis.), AKos Consulting and Solutions GmbH (Basel, Switzerland), Ambinter (Paris, France), Asinex (Moscow, Russia), Aurora (Graz, Austria), BioFocus DPI, Switzerland, Bionet (Camelford, UK), ChemBridge, (San Diego, Calif.), ChemDiv, (San Diego, Calif.), Chemical Block Lt, (Moscow, Russia), ChemStar (Moscow, Russia), Exclusive Chemistry, Ltd (Obninsk, Russia), Enamine (Kiev, Ukraine), Evotec (Hamburg, Germany), Indofine (Hillsborough, N.J.), Interbioscreen (Moscow, Russia), Interchim (Montlucon, France), Life Chemicals, Inc. (Orange, Conn.), Microchemistry Ltd. (Moscow, Russia), Otava, (Toronto, ON), PharmEx Ltd. (Moscow, Russia), Princeton Biomolecular (Monmouth Junction, N.J.), Scientific Exchange (Center Ossipee, N.H.), Specs (Delft, Netherlands), TimTec (Newark, Del.), Toronto Research Corp. (North York ON), UkrOrgSynthesis (Kiev, Ukraine), Vitas-M, (Moscow, Russia), Zelinsky Institute, (Moscow, Russia), and Bicoll (Shanghai, China).

Libraries of natural compounds in the form of bacterial, fungal, plant and animal extracts are commercially available or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Several commercial libraries can be used in the screens.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as airway smooth muscle cells) which have a nonfunctional or altered asthma associated gene. These host cell lines or cells are defective at the polypeptide level. The host cell lines or cells are grown in the presence of drug compound. The rate of constriction or relaxation of the host cells is measured to determine if the compound is capable of regulating the airway responsiveness in the defective cells. Host cells contemplated for use in the present invention include but are not limited to bacterial cells, fungal cells, insect cells, mammalian cells, and plant cells. The asthma-associated SNP encoding DNA molecules may be introduced singly into such host cells or in combination to assess the phenotype of cells conferred by such expression. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

A wide variety of expression vectors are available that can be modified to express the novel DNA sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif 92121); pcDNA3.1/V5&His (Invitrogen); baculovirus vectors such as pVL1392, pVL1393, or pAC360 (Invitrogen); and yeast vectors such as YRP17, YIPS, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the ga14 inducible promoter and the PGK constitutive promoter. In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the asthma-associated SNPs of the present invention or functional fragments thereof provide a system in which to screen potential compounds or agents for the ability to modulate the development of asthma. Thus, in one embodiment, the nucleic acid molecules of the invention may be used to create recombinant cell lines for use in assays to identify agents which modulate aspects of aberrant cytokine signaling associated with asthma and aberrant bronchoconstriction. Also provided herein are methods to screen for compounds capable of modulating the function of proteins encoded by SNP containing nucleic acids.

Another approach entails the use of phage display libraries engineered to express fragment of the polypeptides encoded by the SNP containing nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore.

Thus, one may design drugs which have, e.g., improved polypeptide activity or stability or which act as inhibitors, agonists, antagonists, etc. of polypeptide activity. By virtue of the availability of SNP containing nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of asthma-associated SNP containing nucleic acids enables the production of strains of laboratory mice carrying the asthma-associated SNPs of the invention. Transgenic mice expressing the asthma-associated SNP of the invention provide a model system in which to examine the role of the protein encoded by the SNP containing nucleic acid in the development and progression towards asthma. Methods of introducing transgenes in laboratory mice are known to those of skill in the art. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will facilitate the molecular elucidation of the role that a target protein plays in various processes associated with the asthmatic phenotype, including: aberrant bronchoconstriction, airway inflammation and altered IgE production. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene. Such altered or foreign genetic information would encompass the introduction of asthma-associated SNP containing nucleotide sequences.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use isolated asthma-associated SNP genes as insertional cassettes to selectively inactivate a wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻³. Nonhomologous plasmid-chromosome interactions are more frequent occurring at levels 10⁵-fold to 10² fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformed cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodou-racil, (FIAU). By this counter selection, the number of homologous recombinants in the surviving transformed cells can be increased. Utilizing asthma-associated SNP containing nucleic acid as a targeted insertional cassette provides means to detect a successful insertion as visualized, for example, by acquisition of immunoreactivity to an antibody immunologically specific for the polypeptide encoded by asthma-associated SNP nucleic acid and, therefore, facilitates screening/selection of ES cells with the desired genotype.

As used herein, a knock-in animal is one in which the endogenous murine gene, for example, has been replaced with human asthma-associated SNP containing gene of the invention. Such knock-in animals provide an ideal model system for studying the development of asthma.

As used herein, the expression of a asthma-associated SNP containing nucleic acid, fragment thereof, or an asthma-associated SNP fusion protein can be targeted in a “tissue specific manner” or “cell type specific manner” using a vector in which nucleic acid sequences encoding all or a portion of asthma-associated SNP are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific proteins are well known in the art and described herein.

The nucleic acid sequence encoding the asthma-associated SNP of the invention may be operably linked to a variety of different promoter sequences for expression in transgenic animals. Such promoters include, but are not limited to airway cell specific promoters, a CMV promoter, a prion gene promoter such as hamster and mouse Prion promoter (MoPrP), described in U.S. Pat. No. 5,877,399 and in Borchelt et al., Genet. Anal. 13(6) (1996) pages 159-163; a rat neuronal specific enolase promoter, described in U.S. Pat. Nos. 5,612,486, and 5,387,742; a platelet-derived growth factor B gene promoter, described in U.S. Pat. No. 5,811,633; a brain specific dystrophin promoter, described in U.S. Pat. No. 5,849,999; a Thy-1 promoter; and a PGK promoter; for the expression of transgenes in airway smooth muscle cells.

Methods of use for the transgenic mice of the invention are also provided herein. Transgenic mice into which a nucleic acid containing the asthma-associated SNP or its encoded protein have been introduced are useful, for example, to develop screening methods to screen therapeutic agents to identify those capable of modulating the development of asthma.

### Pharmaceuticals and Methods of Treatment and Uses

In some embodiments, methods for treating asthma are provided comprising administering an agent useful in the treatment of asthma to a subject having one or more SNPs recited in Table 1, or a SNP in linkage disequilibrium with one or more of these SNPs.

In some embodiments, methods for treating asthma in a subject comprising administering an agent useful in the treatment of asthma to a subject having one or more SNPs described herein or a SNP in linkage disequilibrium with one or more of these SNPs.

In some embodiments, the agent comprises one or more of the agents recited in Table 4.

In some embodiments, the agent is selected from one or more of a PGE synthetic agonist, an oral steroid, an anti-IgE, a 131 agonist, a 132 agonist, a mast cell stabilizer, a leukotriene antagonist, Ipratropium bromide, and a phosphodiesterase inhibitor.

In some embodiments, the agent is selected from Epoprostenol, Iloprost, Treprostinil, Methylprednisolone, Prednisone, Prednisolone, Triamcinolone, Omalizumab, Beclomethasone, Budesonide, Ciclesonide, Flunisolide, Fluticasone, Fluticasone propionate HFA, Fluticasone Propionate inhaled, Momethasone, Triamcinolone Acetonide, Triamcinolone, Dobutamine, Epinephrine, Racepinephrine Isoproterenol .beta.1, Isoproterenol .beta.2, Methylxanthine, Theophylline, Arformoterol, Albuterol, Albuterol Sulfate, Clenbuterol, Fenoterol, Formoterol, Isoetarine, Levalbuterol, Levalbuterol HCL, Levalbuterol Tartrate, Metaproterenol, Pirbuterol, Procaterol, Ritodrine, Salmeterol, Terbutaline, Cromolyn, Cromolyn Sodium, Nedocromi, Montelukast, Zafirlukast, Zileuton, Ipratropium Bromide, Aerovent, Apovent, Atrovent, Ipraxa, and Ibudilast.

In each of the method of treating embodiments described above, the method may further comprise administering a second agent that is the same or different from the first agent, each agent being any agent known to those of skill to be useful in the treatment of asthma, such as, for example, the agents of Table 4. In some embodiments, the second agent is selected from

i) a PGE-agonist and a leukotriene inhibitor;

ii) a PGE-agonist and low dose inhaled steroid;

iii) a PGE-agonist and a beta adrenergic agonist;

iv) a PGE-agonist and a phosphodiesterase inhibitor;

v) a PGE-agonist and an anti-IgE antibody;

vi) a PGE-agonist and anticholinergic agent; and

vii) a PGE-agonist and a mast cell stabilizer.

The second agent may be administered at the same time or after the first agent.

In some embodiments, a third agent is administered. In some aspects, the third agent is a mast cell stabilizer. The third agent may be administered at the same time or after the first and/or second agent.

In some embodiments, the PGE-agonist is selected from epoprostenol, iloprost and treprostinil, said leukotriene inhibitor is montelukast; said inhaled steroid is fluticasone; said phospdiesterase inhibitor is theophylline, said anti-IgE antibody is Xolair, said anticholinergic agent is Atrovent, and said mast cell stabilizer is chromolyn.

These agents may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, aerosolized, intramuscular, and intraperitoneal routes.

A lipid nanoparticle composition is a composition comprising one or more biologically active molecules independently or in combination with a cationic lipid, a neutral lipid, and/or a polyethyleneglycol-diacylglycerol (i.e., polyethyleneglycol diacylglycerol (PEG-DAG), PEG-cholesterol, or PEG-DMB) conjugate. In one embodiment, the biologically active molecule is encapsulated in the lipid nanoparticle as a result of the process of providing and aqueous solution comprising a biologically active molecule of the invention (i.e., siRNA), providing an organic solution comprising lipid nanoparticle, mixing the two solutions, incubating the solutions, dilution, ultrafiltration, resulting in concentrations suitable to produce nanoparticle compositions.

Nucleic acid molecules can be administered to cells by incorporation into other vehicles, such as biodegradable polymers, hydrogels, cyclodextrins. (see for example Gonzalez et al., 1999, Bioconjugate Chem., 10, 1068-1074; Wang et al., International PCT publication Nos. WO 03/47518 and WO 03/46185), poly(lactic-co-glycolic)acid (PLGA) and PLCA microspheres (see for example U.S. Pat. No. 6,447,796 and US Patent Application Publication No. US 2002130430), biodegradable nanocapsules, and bioadhesive microspheres, or by proteinaceous vectors (O'Hare and Normand, International PCT Publication No. WO 00/53722)

The following examples are provided to facilitate the practice of the present invention.

### EXAMPLE I

As discussed above, asthma is one of the most common chronic conditions in children. However, despite the importance of asthma in pediatrics most genetic studies carried out to date have been performed in adults of European ancestry. As such, the basis of early onset asthma remains poorly understood, particularly in individuals of non-European ancestry.

To identify genetic determinants of early onset asthma we performed whole genome sequencing of 2113 African American (AA) asthmatics with recurrent exacerbations vs. 2081 AA control samples in our CAG biobank. We performed association testing using generalized linear mixed models (GLMM) in GMMAT. Score tests were generated for each variant followed by Wald tests to estimate effect sizes. Our association analysis identified multiple genome wide significant SNPs at a novel locus on chr7p15.3 that has not been previously associated with asthma.

The association was replicated in an independent cohort of early onset AA asthmatics from the NIH funded TopMed program, including 2088 cases with asthma onset≤12 years vs andb1834 controls. The associated variants mapped to an intergenic region upstream of DNAH11 (rs2529168, disc P 4.7×10⁻⁸, OR 1.47; rep P 0.018; chr7:21315470 (GRCh38.p12)). As mentioned above, DNAH11 encodes a ciliary dynein protein that is involved in the movement of respiratory cilia. Recessive LOF mutations in DNAH11 result in primary ciliary dyskinesia which is characterized by bronchiectasis and upper respiratory tract infections. Rare variant analysis in DNAH11 identified 17 alleles in the cohort distributed between 12 rare variants all of unknown significance. Burden and SKAT tests were not significant, indicating these rare variants were unlikely to be driving the association and hence, are not explaining the association results captured by the common variants at this locus. Taken together, our results demonstrate that common variants at 7p15.3 in WGS data from AA children with early onset asthma and recurrent exacerbations, associate with the disease, a novel locus which has not been previously reported in larger GWAS of asthma in European ancestry adults. The biological function of this gene is of high relevance to asthma and functional studies are underway to establish the exact disease causing mechanism and search for interventions that ameliorate the impact of these disease-causing variants in early onset asthma. We anticipate that the associated variants are capturing a regulatory element, eQTL signal or an enhancer that is responsible for the pathogenic asthma risk effects.

TABLE 1
Mean allele frequencies of the associated SNPs at the DNAH11 locus,
in both the discovery (chop cases/chop control) and replication
(replication cases/control) cohorts with corresponding P values as shown.

| | | | CHOP | CHOP | | |
|---|---|---|---|---|---|---|
| | Replication | Replication | discovery | discovery | Disc | Rep |
| SNP | Cases | Controls | cases | controls | Pval | Pval |
| rs2529136 | 0.15 | 0.14 | 0.15 | 0.13 | 2.46E−05 | 0.013 |
| rs2429063 | 0.12 | 0.11 | 0.13 | 0.09 | 1.37E−06 | 0.044 |
| rs2529155 | 0.13 | 0.12 | 0.14 | 0.10 | 2.63E−07 | 0.153 |
| 7:21303293 | 0.12 | 0.11 | 0.13 | 0.09 | 1.97E−06 | 0.058 |
| rs2529168 | 0.14 | 0.12 | 0.15 | 0.11 | 4.72E−08 | 0.019 |
| rs2700292 | 0.12 | 0.11 | 0.14 | 0.10 | 3.08E−07 | 0.044 |
| rs2700296 | 0.13 | 0.12 | 0.14 | 0.10 | 7.60E−08 | 0.025 |
| 7:21328865 | 0.13 | 0.12 | 0.14 | 0.10 | 4.68E−08 | 0.021 |
| rs10267234 | 0.12 | 0.11 | 0.14 | 0.10 | 9.29E−08 | 0.043 |
| rs150512506 | 0.13 | 0.11 | 0.14 | 0.10 | 9.05E−08 | 0.032 |
| rs28840812 | 0.13 | 0.13 | 0.14 | 0.11 | 1.38E−07 | 0.039 |
| rs111933649 | 0.14 | 0.13 | 0.15 | 0.11 | 1.57E−07 | 0.022 |
| rs78748801 | 0.13 | 0.12 | 0.14 | 0.11 | 7.46E−08 | 0.031 |

As shown, the association replicated in the TopMed dataset in subjects of AA ancestry, including 2088 cases vs 1,800 controls age 12 and under, combined with873 adult controls and 927 adult onset asthma.
Primary ciliary dyskinesia (PCD) is a genetic disorder causing chronic oto-sino-pulmonary disease.
Autosomal recessive mutations in DNAH11
1 case diagnosed with PCD in EHR
3 patients with situs inversus in EHR
SMMAT (Variant Set Mixed Model Association Tests) indicated the association was not driven by DNAH11 rare variants

In additional studies, we have obtained multiple nasal turbinate specimens (left over tissue from turbinectomies performed at the VA hospital of HUP) as a source of ciliated nasal epithelial cells. DNA from these samples has been isolated and genotyped and cell lines that are homozygous major and minor alleles for the associated SNP, rs2529168, were then used to create ciliated primary cell lines to assess ciliary function as a variation of the SNP genotype states. We have currently generated multiple homozygous major allele and heterozygous SNP carrier lines, and we have also identified patients who are homozygous for the minor allele.

DNAH11 constitutes the dynein outer arm of motile cilia and plays a key role in mucociliary clearance within the respiratory tract (also expressed early in ciliogenesis). Our ongoing studies are directed at examining the modulatory effects of DNAH11 on the inflammatory responses observed in the bronchial epithelium in patents with asthma in comparison with healthy controls, addressing the differential effects of the risk allele in this model between cases and controls.

The effects of homozygosity for the risk allele on ciliary beat frequency and ciliary waveform can be determined by isolating ciliated nasal epithelial cells with all 3 genotype states, and using video microscopy to assess the influence of the rs52529168 SNP allele. The cell lines can also be used in advantage in screening assays to identify agents which modulate (i.e., increase or decrease) ciliary beat frequency and ciliary waveform.

Homozygosity for the risk allele may also impact inflammatory cytokine release from nasal and bronchial epithelial cells. Ciliated nasal epithelial cells with all 3 genotype states were isolated and levels of the pro-inflammatory cytokines IL4, IL5, IL6, IL8, IL9, IL13, TNFA and MCPI measured. We anticipate that cytokine levels can vary by genotype.

To determine the effect of homozygosity for the risk allele on inflammatory cell activation and cytokine release, we differentiated peripheral blood mononuclear cells obtained from our patient cohort into macrophages, eosinophils and mast cells and determined the effects of the risk allele on cytokine secretion and cell activation.

In other assays, real time PCR and splicing assays are employed to determine whether homozygosity for the risk allele influences expression or splicing of DNAH11 to better understand the disease causing effects of DNAH11 in the pathophysiology and genetics of asthma, thereby identifying and optimizing therapeutic options for this common and highly morbid condition.

### EXAMPLE II

### Whole Genome Sequencing Identifies Rare Variants Associated with Pulmonary Function and Environmental Pollution in African American Asthmatics

Forced expiratory volume in the first second of exhalation (FEV₁) provides a baseline for lung function and is diagnostic of numerous lung diseases. FEV₁ has been shown to be influenced by both environmental and genetic factors. GWAS of spirometric measures have identified several common variant loci associated with FEV₁ such as the HHIP locus. Here, we sought to identify rare genetic determinants of FEV₁ in a discovery cohort of 1464 African American (AA) asthmatics with recurrent exacerbations from the Center for Applied Genomics (CAG) biobank at CHOP. All patients had asthma diagnosis confirmed by pulmonary/allergy specialists and were using asthma medications (albuterol and inhaled steroids) for at least 6 months. Whole genome sequencing (WGS) data was generated on all samples through the Trans-Omics for Precision Medicine (TOPMed) program. The replication cohort consisted of 876 AA children with asthma from the GALA cohort that were also sequenced through TOPMed.

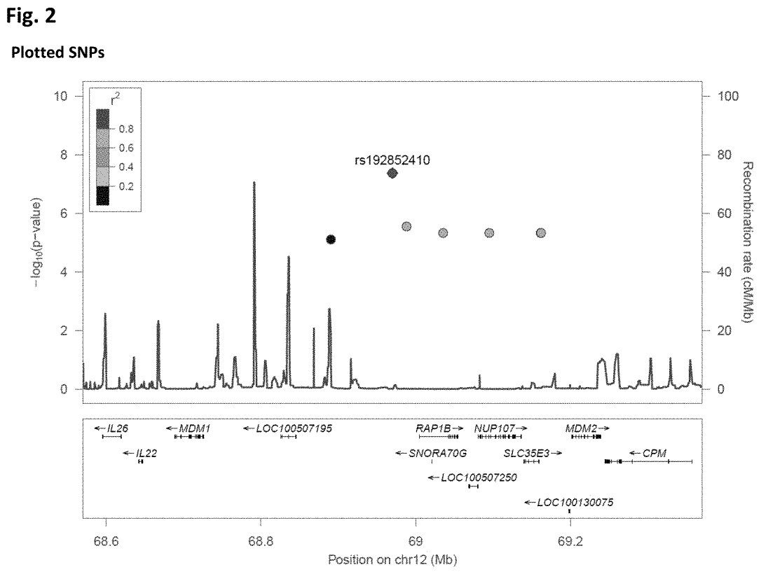

Association testing was performed using the Saige linear model for quantitative traits with kinship adjustment as implemented on the TOPMed Encore server including the first three PCs, age, sex and BMI as covariates. The analysis identified a novel genome wide significant locus on chr12q15 (top SNP rs192852410 P-val 4.29×10⁻⁸, MAF 0.019; beta 0.536) downstream from IL22, IL26 and IFNG. See FIG. 2. The variant association was replicated in WGS data from an independent cohort, the GALA study (rs192852410 Pval 0.02). An IFNG intergenic rare variant that maps to the same locus (rs183884080 Pval 1.6×10^{×6}, MAF 0.0011) was also reported to be associated with FEV₁, only in African Americans, from the in the COPDGene GWAS study (Lutz et al. BMC Genet. (2015) Vol. 16, page 138).

TABLE 2
Mean allele Frequencies and beta and pvalues of rare variants

| | | | Effect | | | | |
|---|---|---|---|---|---|---|---|
| CHR | SNP | BP (hg38) | Allele | Beta | Stat | Pval | MAF |
| 12 | rs12299028 | 68496532 | G | 0.1804 | 4.492 | 7.62E−06 | 0.0234 |
| 12 | rs192852410 | 68576131 | A | 0.536 | 5.508 | 4.29E−08 | 0.0120 |
| 12 | rs145064303 | 68594341 | T | 0.4071 | 4.709 | 2.73E−06 | 0.0157 |
| 12 | rs189759151 | 68641745 | C | 0.3932 | 4.595 | 4.71E−06 | 0.0161 |
| 12 | rs181086557 | 68701358 | C | 0.3932 | 4.595 | 4.71E−06 | 0.0161 |
| 12 | rs142816400 | 68767802 | C | 0.3932 | 4.595 | 4.71E−06 | 0.0161 |
| 12 | rs144961519 | 68768319 | G | 0.3932 | 4.595 | 4.71E−06 | 0.0161 |
| 12 | rs78046756 | 69009578 | T | 0.1407 | 3.996 | 6.77E−05 | 0.0066 |
| 12 | rs116513973 | 69476046 | T | 0.3949 | 4.526 | 6.49E−06 | 0.0059 |
| 12 | rs115656979 | 69594768 | T | 0.3639 | 4.259 | 2.18E−05 | 0.0060 |
| 12 | rs147019971 | 69611987 | G | 0.3469 | 4.014 | 6.27E−05 | 0.0059 |
| 12 | rs74102922 | 69638433 | A | 0.2414 | 4.532 | 6.33E−06 | 0.0154 |
| 12 | rs74102924 | 69639662 | T | 0.2375 | 4.474 | 8.26E−06 | 0.0154 |
| 12 | rs74102926 | 69639668 | T | 0.2375 | 4.474 | 8.26E−06 | 0.0154 |
| 12 | rs74102933 | 69644406 | A | 0.2193 | 3.967 | 7.63E−05 | 0.0140 |
| 12 | rs74585484 | 69649174 | A | 0.2193 | 3.967 | 7.63E−05 | 0.0140 |

We also carried out a gene by environment analysis using geocoding derived air pollution measures including O₃, NO₂, SO₂, PM_{2.5} and PM₁₀. Analyses were conducted as previously described including individual air pollution measures as covariates in the model. The gene-by-environment interaction analyses demonstrated an interaction between the chr12q15 variants, atmospheric SO₂ levels and FEV₁ (rs192852410, Pval 1.971×10⁻⁸, beta 0.54).

This study identified a novel rare variant that maps to a locus containing three genes that have previously been implicated in immune/inflammatory conditions, IL22, IL26 and IFNG in African American children with asthma. GxE analysis indicates the identified variants are also involved in gene-by-SO₂ interaction on FEV₁.

### EXAMPLE III

### Diagnostic Methods for Asthma and Screening Assays to Identify Therapeutic Agents Useful for the Treatment of the Same

The information herein above can be applied clinically to patients for diagnosing an increased susceptibility for developing asthma, and therapeutic intervention. A preferred embodiment of the invention comprises clinical application of the information described herein to a patient. Diagnostic compositions, including microarrays, and methods can be designed to identify the genetic alterations described herein in nucleic acids from a patient to assess susceptibility for developing asthma. This can occur after a patient arrives in the clinic; the patient has blood drawn, and using the diagnostic methods described herein, a clinician can detect the SNPs in the chromosomal regions described herein. The information obtained from the patient sample, which can optionally be amplified prior to assessment, will be used to diagnose a patient with an increased or decreased susceptibility for developing asthma. Kits for performing the diagnostic method of the invention are also provided herein. Such kits comprise a microarray comprising at least one of the SNPs provided herein in and the necessary reagents for assessing the patient samples as described above.

Once a patient has been identified as asthmatic, one or more of the agents listed in Table 3 can be administered.

TABLE 3
Asthma-related medications by subtype used for case inclusion and control
exclusion by the asthma algorithm.Compounds are present by the generic name and the
brand name in parenthesis

Oral Steroids
Methylprednisolone (Medrol, Medrol Dosepak)
Prednisone (Orasone)
Prednisolone (Orapred, Pediapred, Prelone)
Triamcinolone (Aristocort)
Anti IgE: Omalizumab (Xolair)
Inhaled Steroids
Beclomethasone (QVAR)
Budesonide (Pulmicort Turbuhaler, Pulmicort Respules)
Ciclesonide
Flunisolide (Aerobid, Pulmicort Respules)
Fluticasone (Flovent), Fluticasone propionate HFA, Fluticasone Propionate
inhaled
Momethasone (Asmanex Twisthaler)
Triamcinolone (Azmacort), Triamcinolone Acetonide
β1 agonists
Dobutamine
Epinephrine
Racepinephrine
Isoproterenol (β1 and (β2)
Methylxanthine
Theophylline (aminophylline, Dimethylxanthine, Sbo-Bid, Theo, Theodur,
Theolair, Uniphyl)
Xamoterol
β2 agonists
Arformoterol (Brovana, Sunovion, )
Albuterol, Albuterol Sulfate (Salbutamol, Ventolin, Proventil, ProAir,
Accuneb)
Clenbuterol
Epinephrine
Fenoterol
Formoterol (Foradil)
Isoetarine
Isoproterenol (β1 and (β2)
Levalbuterol, Levalbuterol HCL, Levalbuterol Tartrate (Levosalbutamol,
Xopenex)
Metaproterenol
Pirbuterol (Maxair)
Procaterol
Ritodrine
Salmeterol (Serevent)
Terbutaline (Brethine)
Mast cell stabilizers
Cromolyn (Cromoglicic acid, cromoglycate, cromoglicate, Intal),
Cromolyn Sodium
Nedocromil (Alocril, Rapitil, Tilade)
Leukotriene-Antagonists
Montelukast
Zafirlukast
Zileuton
Ipratropium Bromide
Aerovent
Apovent
Atrovent
Ipraxa
Phosphodiesterase Inhibitors: Ibudilast

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for identifying a human subject of African descent as having a predisposition for asthma, comprising,
a) obtaining a nucleic acid sample from said subject;
b) detecting whether the nucleic acid has one or more single nucleotide polymorphisms (SNPs) listed in Table 1 and present in the locus encoding DNA11H, or a SNP in linkage disequilibrium with one or more of the SNPs, by contacting the nucleic acid sample with a probe or primer of sufficient length and composition to detect the SNP; and
c) identifying the subject as having a predisposition for asthma if one or more SNPs are identified.

2. A method for identifying a human subject of African descent as having a predisposition for asthma, comprising,
a) obtaining a nucleic acid sample from said subject;
b) detecting whether the nucleic acid has one or more single nucleotide polymorphisms (SNPs) listed in Table 2 and present in the IL22, IL26 and Interferon gamma locus encoding, or a SNP in linkage disequilibrium with one or more of the SNPs, by contacting the nucleic acid sample with a probe or primer of sufficient length and composition to detect the SNP; and
c) identifying the subject as having a predisposition for asthma if one or more SNPs are identified.

3. The method of claim 1, further comprising administering at least one an agent useful to treat asthma.

4. The method of claim 3, wherein said agent is selected from one or more of a PGE synthetic agonist, an oral steroid, an anti-IgE, a β1 agonist, a β2 agonist, a mast cell stabilizer, a leukotriene antagonist, Ipratropium bromide, and a phosphodiesterase inhibitor.

5. The method of claim 3, wherein said agent is selected from Epoprostenol, Iloprost, Treprostinil, Methylprednisolone, Prednisone, Prednisolone, Triamcinolone, Omalizumab, Beclomethasone, Budesonide, Ciclesonide, Flunisolide, Fluticasone, Fluticasone propionate HFA, Fluticasone Propionate inhaled, Momethasone, Triamcinolone Acetonide, Triamcinolone, Dobutamine, Epinephrine, Racepinephrine Isoproterenol β1, Isoproterenol β2, Methylxanthine, Theophylline, Arformoterol, Albuterol, Albuterol Sulfate, Clenbuterol, Fenoterol, Formoterol, Isoetarine, Levalbuterol, Levalbuterol HCL, Levalbuterol Tartrate, Metaproterenol, Pirbuterol, Procaterol, Ritodrine, Salmeterol, Terbutaline, Cromolyn, Cromolyn Sodium, Nedocromi, Montelukast, Zafirlukast, Zileuton, Ipratropium Bromide, Aerovent, Apovent, Atrovent, Ipraxa, and Ibudilast.

6. The method of claim 3, wherein a combination of agents is administered, said combination selected from i) a PGE-agonist and a leukotriene inhibitor; ii) a PGE-agonist and low dose inhaled steroid; iii) a PGE-agonist and a beta adrenergic agonist; iv) a PGE-agonist and a phosphodiesterase inhibitor; v) a PGE-agonist and an anti-IgE antibody; vi) a PGE-agonist and anticholinergic agent; and vii) a PGE-agonist and a mast cell stabilizer.

7. The method of claim 6, wherein combinations i-vi further comprise a mast cell stabilizer.

8. The method of claim 6, wherein said PGE-agonist is selected from epoprostenol, iloprost and treprostinil, said leukotriene inhibitor is montelukast; said inhaled steroid is fluticasone; said phospdiesterase inhibitor is theophylline, said anti-IgE antibody is Xolair, said anticholinergic agent is Atrovent, and said mast cell stabilizer is chromolyn.

9.-13. (canceled)

14. A method for diagnosing and treating asthma in a human subject of African descent, comprising,
a) detecting whether at least one single nucleotide polymorphism (SNP), rs2529168 is present in a nucleic acid sample from the subject,
b) diagnosing the subject with asthma when the presence of at least one SNP is detected; and
c) administering an effective amount of an agent useful for the treatment of asthma.

15. A method for diagnosing and treating asthma in a human subject of African descent, comprising,
a) detecting whether at least one single nucleotide polymorphism (SNP), rs19285410 is present in a nucleic acid sample from the subject,
b) diagnosing the subject with asthma when the presence of at least one SNP is detected; and
c) administering an effective amount of an agent useful for the treatment of asthma.

16. The method of claim 14, wherein the agent is one or more of the agents described in Table 3.

17.-20. (canceled)

21. A method of treating asthma in a patient having any one or more of the SNPs of claim 1, comprising administering one or more agents useful in treating asthma.

22. The method of claim 21, wherein the agent is listed in Table 3.

23. (canceled)

24. The method of claim 2, further comprising administering at least one an agent useful to treat asthma.

25. The method of claim 15, wherein the agent is one or more of the agents described in Table 3.

26. A method of treating asthma in a patient having any one or more of the SNPs of claim 2, comprising administering one or more agents useful in treating asthma.

27. The method of claim 26, wherein the agent is listed in Table 3.
